(19) United States
(12) Patent Application Publication
(10) Pub. No.: US 2017/0351121 A1
(43) Pub. Date: Dec. 7, 2017

(54) GLASSES FRAME STRUCTURE

(71) Applicant: Yeou-Bang WANG, Tainan (TW)

(72) Inventor: Yeou-Bang WANG, Tainan (TW)

(73) Assignee: Mao-Shen LIN, Taichung (TW)

(21) Appl. No.: 15/427,064

(22) Filed: Feb. 8, 2017

(30) Foreign Application Priority Data
Jun. 3, 2016 (TW) 105208351

(51) Int. Cl. G02C 11/00; G02C 5/14; G02C 1/06; G02C 5/22; A61F 9/02

(52) U.S. Cl. CPC G02C 11/12; G02C 5/22; A61F 9/029; G02C 1/06; G02C 5/146

(57) ABSTRACT

A glasses frame structure includes a chassis, a pair of temples, a pair of hinges, and a plurality of fasteners. The chassis has a pair of front rims and a pair of side rims respectivey next to the front rims. Each of the side rims of the chassis can receive a first side lens. Each temple has a rimmed portion adjacent to one of the side rims of the chassis. The rimmed portion of each of the temples can receive a second side lens. The hinges can pivotally connect the temples to the chassis. Some of the fasteners can fix the hinges and the first side lenses to the chassis, while some of the fasteners can fix the hinges and the second side lenses to the temples. With the glass frame structure, the side lenses can be installed more easily and firmly.

### BACKGROUND OF THE INVENTION

### 1. Field of the Invention

The present invention relates to a glasses frame structure and, more particularly, to a glasses frame structure that can be fitted with side lenses more easily and firmly.

### 2. Description of the Prior Art

Generally, glasses can be classified into two categories: medical corrective glasses and eye protection glasses, according to their functions. Eye protection glasses can be used for protecting human eyes from ultraviolet or intense light due to snow, foreign matter due to chemical sputtering, lathe cutting, milling or drilling, glare of computer display, X-ray, laser, intense light due to welding, dust, or pollen. Also, eye protection glasses can be used for the military use or town violence. With the improvement of quality of life, the increase in outdoor sports, and the popularization of eye medical and protection knowledge, there are various glasses products coming in the market, such as military glasses, sport safety glasses, working protective glasses, chemical protective mask, and motorcycle goggles. People increasingly demand the quality of the glasses products.

In conventional safety glasses, for protecting the eyes of engineering staff, athletes or workers, the glass frames are provided with side structures or shields. The protection level of the safety glasses, which can be determined by using an air gun to strike the lenses of the safety glasses at a specified velocity and a specified pressure several times, is generally divided into low standard (such as the standard required in Japan, wherein the lens to be tested should not fall from the associated frame by a gun fired at a velocity between 90 and 110 miles per hour), intermediate standard (such as the standard required in European countries, wherein the lens to be tested should not fall from the associated frame by a gun fired at a velocity of 120 miles per hour), and high standard (such as the standard required in US, wherein the lens to be tested should not fall from the associated frame by a gun fired at a velocity between 160 and 165 miles per hour). In conventional safety glasses, for increasing the capability of a glasses frame resisting an impact and increasing the safety level of the glasses frame, the side structures or shields thereof are required to be widen and thickened. In one example of conventional safety glasses, as disclosed in Publication Number 255500 and 255733 of Taiwan Patent, the chassis is curved backwardly to provide lateral protection for eyes. In another example of conventional safety glasses, as disclosed in Publication Number 385014 of Taiwan Patent, the chassis defines a V-shaped groove for holding the lenses, wherein the groove is unequal in its two sides, and one side of the groove is provided with one stop edge to increase the safety level of the glasses frame. A further example of the conventional safety glasses, as disclosed in Publication Number D127510 of Taiwan Patent, shows that the chassis defines side rims that can be fitted with side lenses. The conventional safety glasses hold the side lenses only via the side rims. There is still room for improving the conventional safety glasses. A further example of the conventional safety glasses, as disclosed in D138775, shows that the chassis defines slots for mounting the associated lenses.

The applicant has engaged in the processing and manufacturing of glasses for many years. After years of effort and repeated tests to overcome the shortcomings of conventional glasses frames, an enhanced glasses frame is thus invented and disclosed herein.

### SUMMARY OF THE INVENTION

One object of the present invention is to provide an enhanced safety glasses frame, wherein the chassis thereof has a pair of side rims to each receive a first side lens, and each of the temples thereof has a rimmed portion to receive a second side lens. One advantage of the present invention is that the glasses frame can be made in a variety of styles while the side lenses can be made in a standard size to facilitate installation of the side lenses. With the present invention, the side lenses can be fixed more easily and firmly, the safety level of the glasses frame can be increased, and the cost of the glasses frame can be reduced.

Other objects, advantages, and novel features of the present invention will become more apparent from the following detailed description when taken in conjunction with the accompanying drawings.

### BRIEF DESCRIPTION OF THE DRAWINGS

FIG. 1 shows an exploded view of a glasses frame structure according to one embodiment of the present invention.

FIG. 2 shows a side view of the glasses frame structure.

FIG. 3 shows a top view of the glasses frame structure.

FIG. 4 shows a rear view of the chassis of the glasses frame structure.

FIG. 5 shows a front view of the temples of the glasses frame structure.

FIG. 6 shows a schematic application view of the glasses frame structure, wherein two side lenses can be fitted into the chassis and one of the temples respectively.

### DETAILED DESCRIPTION OF THE PREFERRED EMBODIMENTS

The following paragraphs will illustrate one embodiment of the present invention in detail to allow the technical contents, features and advantages of the present invention to be understood more easily.

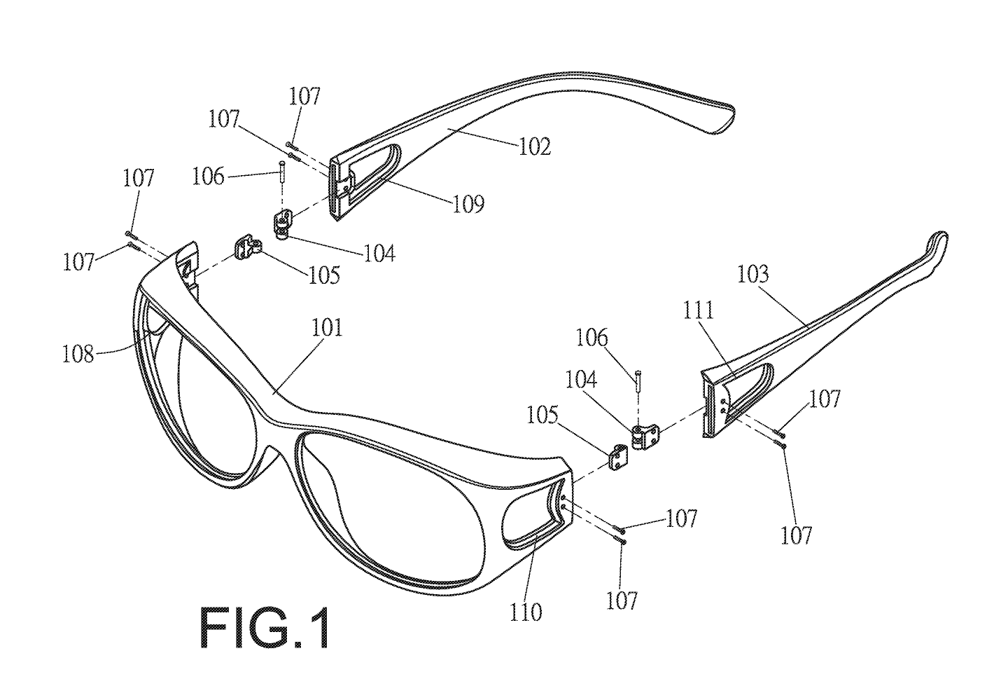

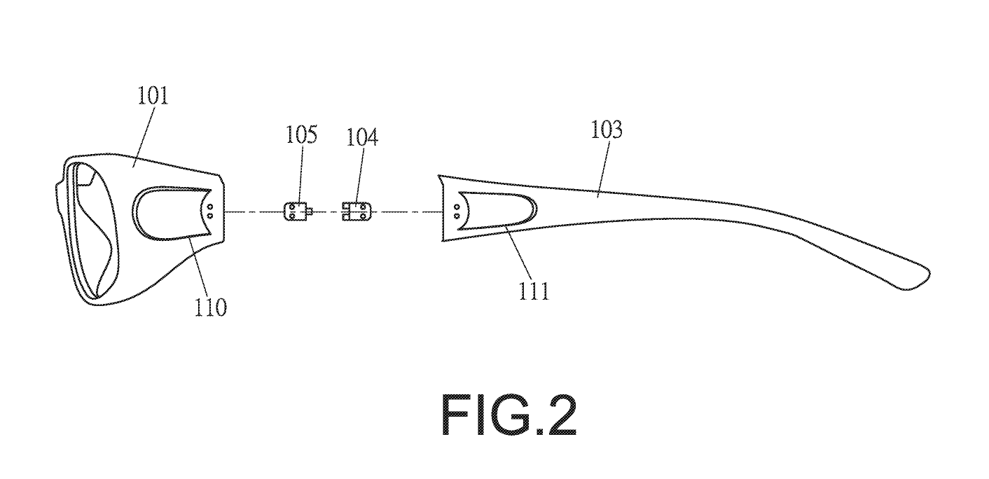

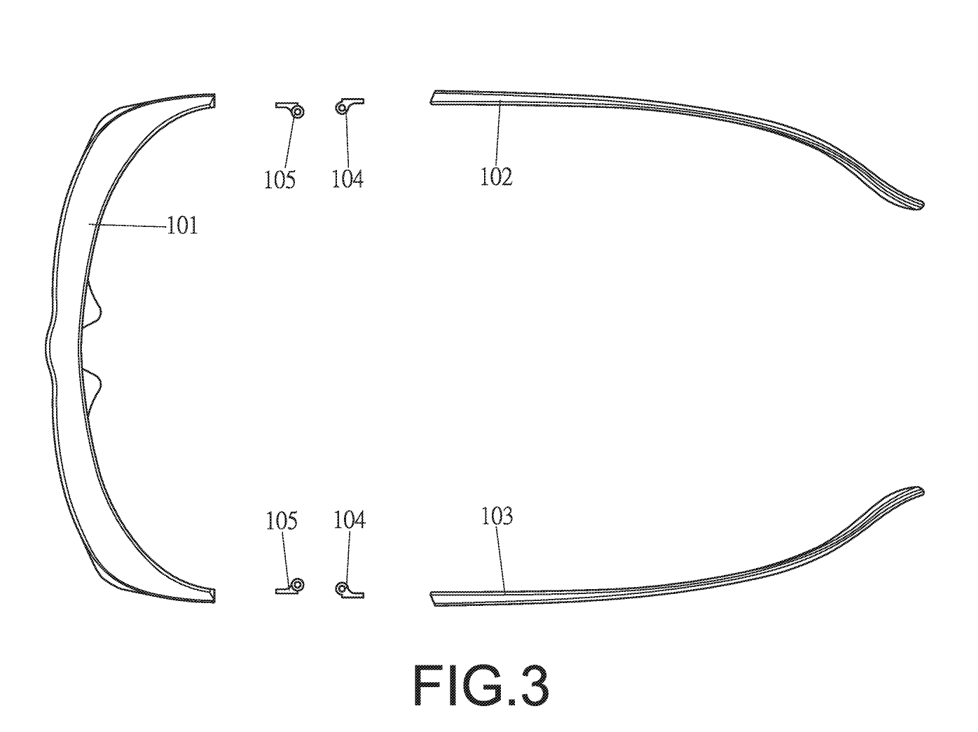

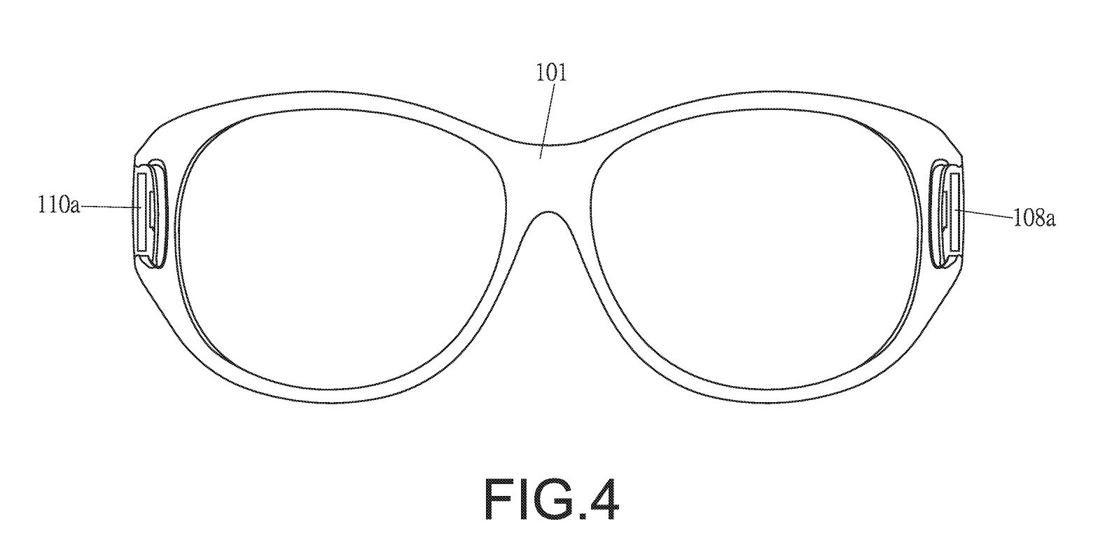

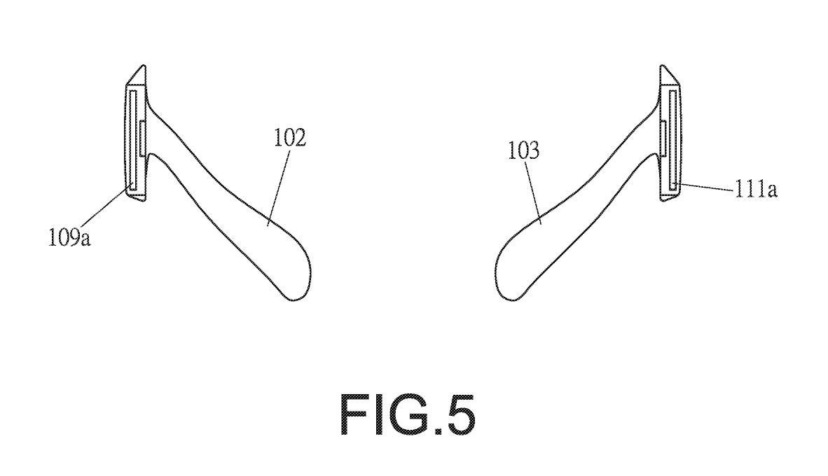

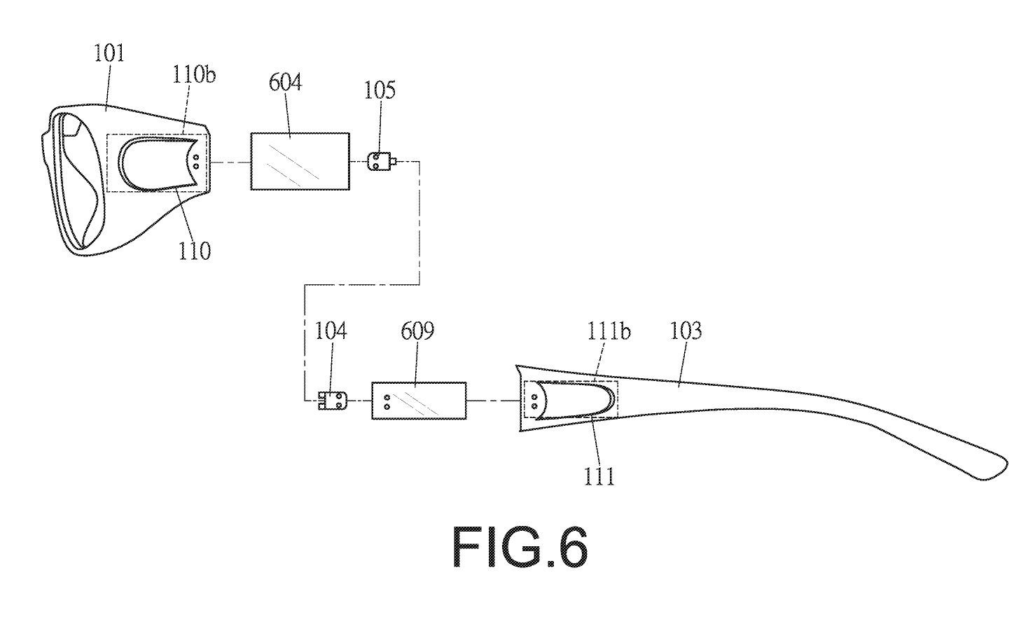

Referring first to FIGS. 1, 2, 3 and 6, a glasses frame structure according to one embodiment of the present invention is shown, which generally comprises a chassis 101, a pair of temples including a right temple 102 and a left temple 103, a pair of hinges each including a first part 105, a second part 104, and a pivot pin 106 that connects the first and second parts 105, 104, and a plurality of fasteners 107, such as screws. One feature of the present invention is that, in addition to a pair of front rims for normal lenses, the chassis 101 has a pair of side rims 108, 110 respectively next to the front rims, and each side rim is extended at an angle to the corresponding front rim. Each of the side rims 108, 110 of the chassis 101 defines a space for receiving a first side lens, which is preferably standardized in size. Also, each of the rimmed portions 109, 111 of the temples 102, 103, which is adjacent to one of the side rimes, defines a space for receiving a second side lens, which is preferably standardized in size. In particular, each of the side rims 108, 110 defines a slot 108a, 110a (see FIG. 4), through which a first side lens can be fitted into the corresponding space; each of the rimmed portions 109, 111 defines a slot 109a, 111a (see FIG. 5), through which a second side lens can be fitted into the corresponding space. As shown in FIGS. 1 and 6, after a first side lens 604 has been fitted into the space 110b of the left side rim 110 of the chassis 101, both the first part 105 of a hinge and the first side lens 604 can be fixed to the chassis 110 by a plurality of fasteners 107. Similarly, after a second side lens 609 has been fitted into the space 111b of the rimmed portion 110 of the left temple 103, both the second part 104 of the hinge and the second side lens 609 can be fixed to the left temple 103 by a plurality of fasteners 107. The pivot pin 106 connects the first part 105 and the second part 104 pivotally. Thus, the hinges can be used to pivotally connect the temples 102, 103 to the chassis 101. It is not necessarily that the side rims 108, 110 of the chassis should coexist with the rimmed portions 109, 111 of the temples. In one embodiment, the side rims 108, 110 of the chassis or the rimmed portions 109, 111 of the temples can be dispensed with. Another feature of the present invention is that one hinge and one side lens can be fixed to the chassis or one temple by same fasteners to reduce the components required for the glasses frame and to increase the safety level of the glasses frame structure.

FIG. 4 shows a rear view of the chassis 101. As shown, the slots 108a, 110a of the side rims are rectangular. However, the slots 108a, 110a can be configured to be curved to conform with a curved side lens. FIG. 5 shows a front view of the temples 102, 103. As shown, the slots 109a, 111a of the rimmed portions of the temples 102, 103 are rectangular. However, the slot 109a, 111a can be configured to be curved to conform with a curved side lens. FIG. 6 shows a schematic application view of the glasses frame structure, wherein the left side rim 110 defines a space 110b for receiving a first side lens 604. The first side lens 604 can be fitted into the space 110b through the slot 110a (see FIG. 4), and the second side lens 609 can be fitted into the space 111b through the slot 111a (see FIG. 5). Thereafter, a plurality of fasteners 107 can be used to firmly fix both the first part 105 of one hinge and the first side lens 604 to the chassis 101, and to firmly fix both the second part 104 of the hinge and the second side lens 609 to the temple 103. The glasses frame structure of the present invention allows side lenses to be fixed more easily and firmly with fewer fasteners, and the cost of the glasses frame can be reduced.

While the invention has been described with reference to the preferred embodiment above, it should be recognized that the preferred embodiment is given for the purpose of illustration only and is not intended to limit the scope of the present invention and that various modifications and changes, which will be apparent to those skilled in the relevant art, may be made without departing from the spirit and scope of the invention.

What is claimed is:

1. A glasses frame structure, comprising:
a chassis having a pair of front rims and a pair of side rims respectively next to the front rims, each of the side rims of the chassis capable of receiving a first side lens;
a pair of temples each having a rimmed portion adjacent to one of the side rims of the chassis, the rimmed portion of each of the temples capable of receiving a second side lens;
a pair of hinges for pivotally connecting the temples to the chassis; and
a plurality of fasteners capable of fixing the hinges and the first side lenses to the chassis and fixing the hinges and the second side lenses to the temples.

2. The glasses frame structure of claim 1, wherein each of the hinges is composed of a first part, a second part, and a pivot pin that connects the first part to the second part, some of the fasteners capable of fixing the first parts of the hinges and the first side lenses to the chassis, some of the fasteners capable of fixing the second parts of the hinges and the second side lenses to the temples.

3. The glasses frame structure of claim 1, wherein each of the side rims of the chassis defines a rectangular or curved slot, through which a first side lens can be fitted into the corresponding side rim of the chassis.

4. The glasses frame structure of claim 1, wherein the rimmed portion of each of the temples defines a rectangular or curved slot, through which a second side lens can be fitted into the rimmed portion of the corresponding temple.

5. A glasses frame structure, comprising:
a chassis having a pair of front rims and a pair of side rims respectively next to the front rims, each of the side rims of the chassis capable of receiving a side lens;
a pair of temples;
a pair of hinges for pivotally connecting the temples to the chassis; and
a plurality of fasteners capable of fixing the hinges and the side lenses to the chassis.
